(19) United States
(12) Patent Application Publication
(10) Pub. No.: US 2009/0023735 A1
(43) Pub. Date: Jan. 22, 2009

(54) Sulphonamide Derivatives

(76) Inventors: Jyrki Heino, Turku (FI); Mark Johnson, Turku (FI); Jarmo Kapyla, Lieto (FI); Anne Marjamaki, Littoinen (FI); Tommi Nyronen, Helsinki (FI); Marika Ojala, Rusko (FI); Olli Pentikainen, Vaajakoski (FI); Marjo Pihlavisto, Kaarina (FI); Liisa Nissinen, Turku (FI)

Correspondence Address: STERNE, KESSLER, GOLDSTEIN & FOX P.L.L.C., 1100 NEW YORK AVENUE, N.W., WASHINGTON, DC, 20005, US

(21) Appl. No.: 12/066,950

(22) PCT Filed: Sep. 15, 2006
(86) PCT No.: PCT/FI06/50395

§ 371 Date: Apr. 17, 2008

(30) Foreign Application Priority Data
Sep. 16, 2005 (FI) 20055498

(51) Int. Cl. A61K 31/18; A61P 7/02; A61P 29/00; A61P 35/00; C07D 261/08; C07C 311/21; A61K 31/4164; A61K 31/44; C07D 295/10; C07D 233/61; C07D 213/52; A61K 31/5377; A61K 31/42

(52) U.S. Cl. 514/238.2; 548/247; 564/84; 544/160; 548/335.5; 546/333; 514/238.8; 514/378; 514/602; 514/399; 514/392; 514/354

(57) ABSTRACT

The invention relates to sulphonamide derivatives of formula (I), where R_{C} is selected from a group consisting of dialkylamino, NO₂, CN, aminocarbonyl, monoalkylaminocarbonyl, dialkylaminocarbonyl, alkanoyl, oxazol-2-yl, oxazolylaminocarbonyl, aryl, aroyl, aryl-CH(OH)—, arylaminocarbonyl, furanyl, where the aryl, aroyl and furanyl moieties may be substituted, guanidinyl-(CH₂)_{z}—N(R′)—, Het-(CH₂)_{z}—N(R′)—, Het-CO—N(R′)—, Het-CH(OH)— and Het-CO—, where Het is an optionally substituted 4-6-membered heterocyclic ring containing one or more heteroatoms sleeted from N, S and O, R′ is hydrogen or alkyl, and z is an integer 1 to 5; R_{A} is a group of formula (A), (B), (C) or (D) as defined in the claims; and R_{B} is hydrogen, alkyl, alkanoyl, hydroxyalkyl, alkoxyalkyl, alkoxycarbonyl, alkoxycarbonylalkyl, aminoalkyl, mono- or dialkylaminoalkyl or Het-alkyl, where Het is as defined above. The invention also relates to the use of derivatives of formula (I) as inhibitors for collagen receptor integrins and a process for preparing sulphonamides of formula (II).

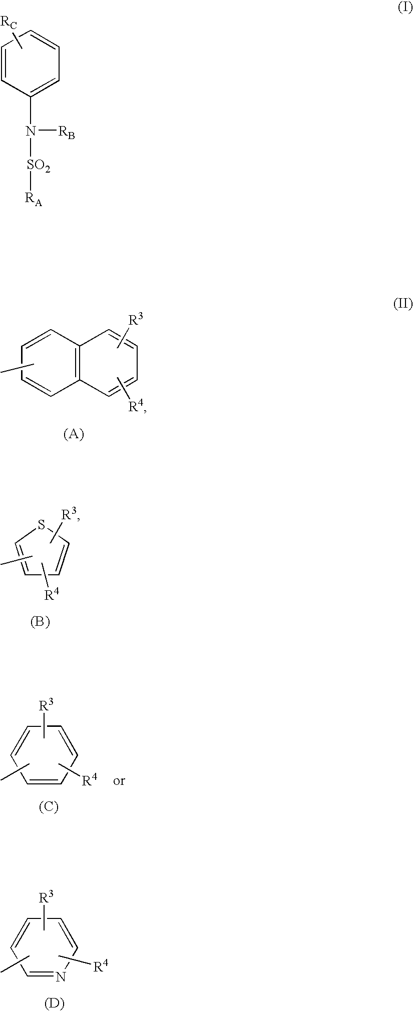

### FIELD OF THE INVENTION

The present invention relates to sulphonamide derivatives of formula (I) and physiologically acceptable salts thereof,

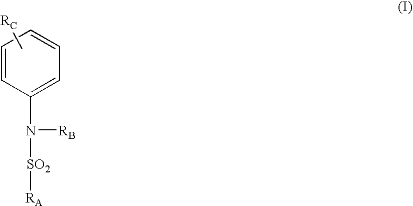

where

R_{C} is selected from a group consisting of dialkylamino, NO₂, CN, aminocarbonyl, monoalkylaminocarbonyl, dialkylaminocarbonyl, alkanoyl, oxazol-2-yl, oxazolylaminocarbonyl, aryl, aroyl, aryl-CH(OH)—, arylaminocarbonyl, furanyl, where the aryl, aroyl and furanyl moieties may be substituted, guanidinyl-(CH₂)_{z}—N(R′)—, Het-(CH₂)_{z}—N(R′)—, Het-CO—N(R′)—, Het-CH(OH)— and Het-CO—, where Het is an optionally substituted 4-6-membered heterocyclic ring containing one or more heteroatoms selected from N, O and S, R′ is hydrogen or alkyl, and z is an integer 1 to 5;

R_{A} is a group having the formula

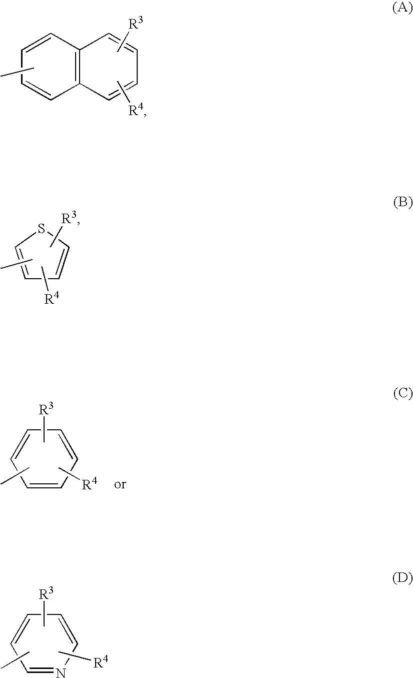

wherein

R³ and R⁴ represent each independently hydrogen, halogen, aryl, alkoxy, carboxy, hydroxy, alkoxyalkyl, alkoxycarbonyl, cyano, trifluoromethyl, alkanoyl, alkanoylamino, trifluoromethoxy, an optionally substituted aryl group, and

R_{B} is hydrogen, alkyl, alkanoyl, hydroxyalkyl, alkoxyalkyl, alkoxycarbonyl, alkoxycarbonylalkyl, aminoalkyl, mono- or dialkylaminoalkyl or Het-alkyl, where Het is as defined above;

provided that
(i) when R_{C} is dialkylamino, then R_{B} is not hydrogen or alkyl;
(ii) when R_{A} is a group of formula (C), where R³ is hydrogen and R₄ is methoxy, then R_{C} is not Het-CO—N(R′)—; and
(iii) when R_{A} is a group of formula (C), where R³ and R⁴ are hydrogen or halogen, then R_{C} is not nitro.

The invention also relates to the use of the derivatives of formula (I) as inhibitors of collagen receptor integrins, especially α2β1 integrin inhibitors and more precisely α2β1 integrin I-domain inhibitors, e.g. in connection with diseases and medical conditions that involve the action of cells and platelets expressing collagen receptors, their use as a medicament, e.g. for the treatment of thrombosis, inflammation, cancer and vascular diseases, pharmaceutical compositions containing them and a process for preparing them.

### BACKGROUND OF THE INVENTION

The integrins are a large family of cell adhesion receptors, which mediate anchoring of all human cells to the surrounding extracellular matrix. In addition integrins participate in various other cellular functions, including cell division, differentiation, migration and survival. The human integrin gene family contains 18 alpha integrin genes and 8 beta integrin genes, which encode the corresponding alpha and beta subunits. One alpha and one beta subunit is needed for each functional cell surface receptor. Thus, 24 different alpha-beta combinations exist on human cells. Nine of the alpha subunits contain a specific “inserted” I-domain, which is responsible for ligand recognition and binding. Four of the α I-domain containing integrin subunits, namely α1, α2, α10 and α11, are the main cellular receptors of collagens. Each one of these four alpha subunits forms a heterodimer with beta1 subunit. Thus the collagen receptor integrins are α1β1, α2β1, α10β1 and α11β1 (Reviewed in White et al., Int J Biochem Cell Biol, 2004, 36:1405-1410). Collagens are the largest family of extracellular matrix proteins, composed of at least 27 different collagen sub-types (collagens l-XXVII).

Integrin α2β1 is expressed on epithelial cells, platelets, inflammatory cells and many mesenchymal cells, including endothelial cell, fibroblasts, osteoblasts and chondroblasts (Reviewed in White et al., supra). Epidemiological evidence connect high expression levels of α2β1 on platelets to increased risk of myocardial infarction and cerebrovascular stroke (Santoso et al., Blood, 1999, Carlsson et al., Blood. 1999, 93:3583-3586), diabetic retinopathy (Matsubara et al., Blood. 2000, 95:1560-1564) and retinal vein occlusion (Dodson et al., Eye. 2003, 17:772-777). Evidence from animal models support the proposed role of α2β1 in thrombosis. Integrin α2β1 is also overexpressed in cancers such as invasive prostate cancer, melanoma, gastric cancer and ovary cancer. These observations connect α2β1 integrin to cancer invasion and metastasis. Moreover, cancer-related angiogenesis can be partially inhibited by anti-α2 function blocking antibodies (Senger et al., Proc. NatI. Acad. Sci. U.S.A., 1997, 94:13612-13617). Finally, leukocytes are partially dependent on α2β1 function during inflammatory process (de Fougerolles et al., J. Clin. Invest., 2000, 105:721-729). Based on the tissue distribution and experimental evidence α1β1 integrin may be important in inflammation, fibrosis, bone fracture healing and cancer angiogenesis (White et al., supra), while all four collagen receptor integrins may participate in the regulation of bone and cartilage metabolism.

The strong evidence indicating the involvement of collagen receptors in various pathological processes has made them potential targets of drug development. Function blocking antibodies against α1 or α2 subunits have been effective in several animal models including models for inflammatory diseases and cancer angiogenesis. Synthetic peptide inhibitors as well as snake venom peptides blocking the function of α1β1, and α2β1 have been described. (Eble, Curr Pharm Design 2005, 11:867-880). International Patent Publication WO 99102551 discloses one small molecule drug candidate that regulates the expression of α2β1 but it is not actually binding to the integrin.

Publication EP 1 258 252 A1 describes certain N-indolyl-, N-quinolinyl-, N-isoquinolinyl- and N-coumarinyl-arylsulphonamides, which are stated to be integrin expression inhibitors. Said publication does not specifically disclose the compounds of the present invention. Further, said known compounds differ from the compounds now described with respect to their properties and the mechanism of function. The compounds of the present invention are not integrin expression suppressors.

Publication EP 0 472 053 B1 discloses sulphonamides having anti-tumor activity. The compounds specifically described in said publication do not fall within the definition of the compound group of the present invention.

Publication lzvestiya Aakademii Nauk SSSR, Seriya Khimicheskaya (1981), (6), Kravtsov, D. N. et al., pp. 1259-1264 discloses sulphonamides, which are structurally closely related to the compounds now described but which do not fall within the definition of the compound group of the present invention. The field of use of the known compounds is totally different from that of the present invention.

Publication WO 2004/005278 discloses bisarylsulphonamides and their use in cancer therapy. Said publication does not specifically describe compounds falling within the definition of the compound group of the present invention.

It has now surprisingly been found that the compounds of formula (I) according to the present invention are potent inhibitors for collagen receptor integrins, especially α2β1 integrin, and may be used in the treatment of human diseases, such as thrombosis, cancer, fibrosis, inflammation and vascular diseases. The compounds of formula (I) may also be used in diagnostic methods both in vitro and in vivo.

### SUMMARY OF THE INVENTION

The present invention relates sulphonamide derivatives of formula (I) and physiologically acceptable salts thereof,

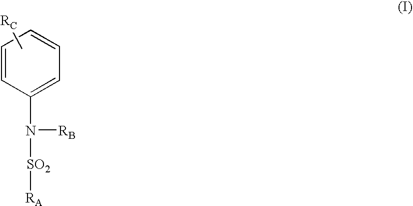

where

R_{C} is selected from a group consisting of dialkylamino, NO₂, CN, aminocarbonyl, monoalkylaminocarbonyl, dialkylaminocarbonyl, alkanoyl, oxazol-2-yl, oxazolylaminocarbonyl, aryl, aroyl, aryl-CH(OH)—, arylaminocarbonyl, furanyl, where the aryl, aroyl and furanyl moieties may be substituted, guanidinyl-(CH₂), —N(R′)—, Het-(CH₂)_{z}—N(R′)—, Het-CO—N(R′)—, Het-CH(OH)— and Het-CO—, where Het is an optionally substituted 4-6-membered heterocyclic ring containing one or more heteroatoms selected from N, O and S, R′ is hydrogen or alkyl, and z is an integer 1 to 5;

R_{A} is a group having the formula

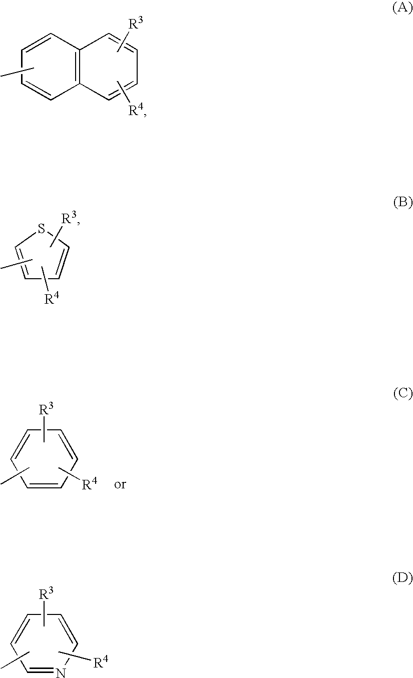

wherein

R³ and R⁴ represent each independently hydrogen, halogen, aryl, alkoxy, carboxy, hydroxy, alkoxyalkyl, alkoxycarbonyl, cyano, trifluoromethyl, alkanoyl, alkanoylamino, trifluoromethoxy, an optionally substituted aryl group, and

R_{B} is hydrogen, alkyl, alkanoyl, hydroxyalkyl, alkoxyalkyl, alkoxycarbonyl, alkoxycarbonylalkyl, aminoalkyl, mono- or dialkylaminoalkyl or Het-alkyl, where Het is as defined above;

provided that
(i) when R_{C} is dialkylamino, then R_{B} is not hydrogen or alkyl;
(ii) when R_{A} is a group of formula (C), where R³ is hydrogen and R⁴ is methoxy, then R_{C} is not Het-CO—N(R′)—; and
(iii) when R_{A} is a group of formula (C), where R³ and R⁴ are hydrogen or halogen, then R_{C} is not nitro.

Further the invention relates to derivatives of formula (I) for use as inhibitors for collagen receptor integrins specifically α2β1 integrin inhibitors and more precisely α2β1 integrin I-domain inhibitors.

The invention also relates to derivatives of formula (I) and physiologically acceptable salts thereof for use as a medicament.

Further the invention relates to the use of a derivative of formula (I) for preparing a pharmaceutical composition for treating disorders relating to thrombosis, inflammation, cancer and vascular diseases.

The present invention also relates to a pharmaceutical composition comprising an effective amount of a derivative of formula (I) or a physiologically acceptable salts thereof in admixture with a pharmaceutically acceptable carrier.

Further the invention relates to a process for preparing benzenesulphonamide derivatives of formula (I) comprising reacting a compound of formula (II),

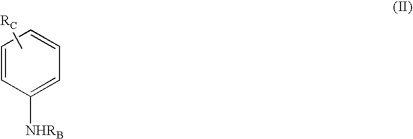

where R_{B} and R_{C} are as defined above, with a compound of formula (III),

R_{A}—SO₂hal (III)

where R_{A} is as defined above and hal is halogen.

### DETAILED DESCRIPTION OF THE INVENTION

In the definition of the compound group of formula (I), typical meanings of the symbol Het, i.e. “an optionally substituted 4-6-membered heterocyclic ring containing one or more heteroatoms selected from N, S and O”, in connection with R_{C} are groups such as oxazol-2-yl, pyrrolyl, pyrazolyl, pyridyl, pyrimidinyl and morfolinyl.

The meaning “alkyl” used herein refers to branched or straight chain alkyl groups having suitably 1 to 6 carbon atoms, preferably 1 to 3 carbon atoms.

The meaning “alkanoyl” refers to branched or straight chain alkyl-carbonyl groups having suitably a total of 1 to 6 carbon atoms, preferably 1 to 3 carbon atoms.

The term “alkoxy” refers to branched or straight chain alkyloxy groups having suitably 1 to 6 carbon atoms, preferably 1 to 3 carbon atoms, in the alkyl moiety.

Examples of “aryl” groups in connection with the definition of R_{C} are phenyl and naphtyl, especially phenyl.

Examples of “aroyl” are benzoyl and naphtoyl, especially benzoyl.

Typical optional substituents in the definitions of R_{C}, R_{A} and R_{B} are halogen, alkyl having 1 to 6 carbon atoms, alkoxy having 1 to 6 carbon atoms, and oxo.

In formulae (A), (B), (C) and (D) R³ and R⁴ are suitably halogen, haloaryl or alkoxyaryl. Examples of R³ and R⁴ having the meaning alkoxyalkyl, alkoxycarbonyl and alkanoyl are those containing 1 to 6 carbon atoms in the alkoxy moiety and 1 to 6 carbon atoms in the alkyl moiety. Examples of optionally substituted aryl groups are

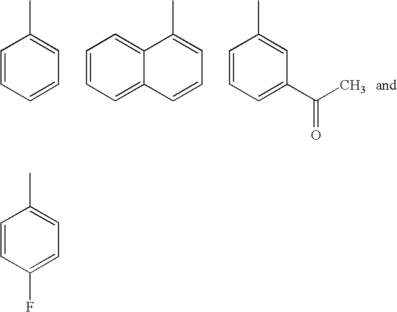

Preferred compounds of formula (I) are those where R_{C} is aroyl or aryl-CH(OH)—, especially benzoyl; R_{B} is hydrogen or alkyl; and R_{A} is a group of formula (C), where R³ and R⁴ are halogens, especially chloro, or R³ is hydrogen and R⁴ phenyl substituted with halogen, especially fluoro.

Typical compounds of the present invention are shown in Table 1.

TABLE 1

| | Compound |
|---|---|
| | number |
| 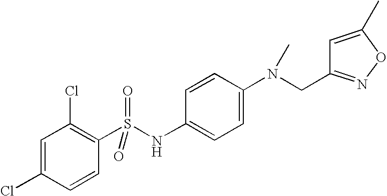 | 329 |
| 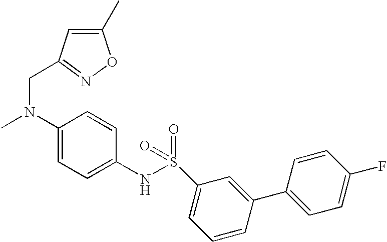 | 343 |
| 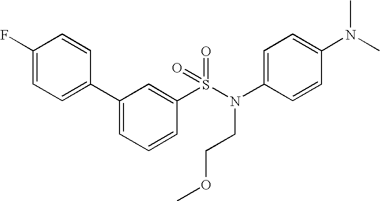 | 353 |
| 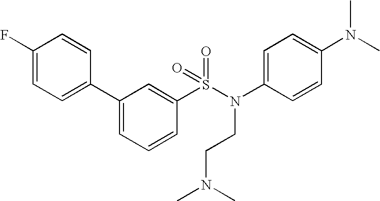 | 354 |
| 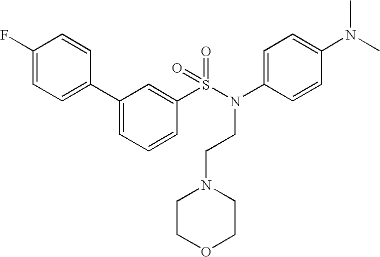 | 355 |
| 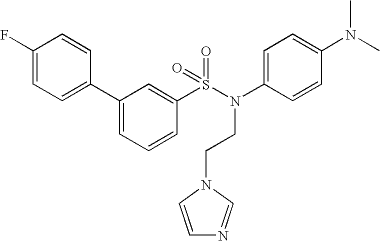 | 358 |
| 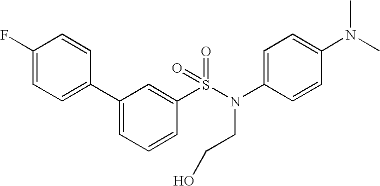 | 359 |
| 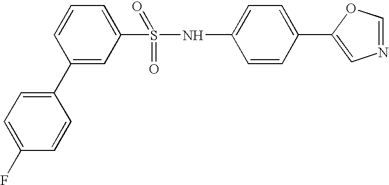 | 378 |
| 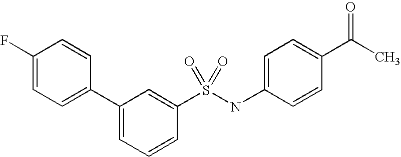 | 383 |
| 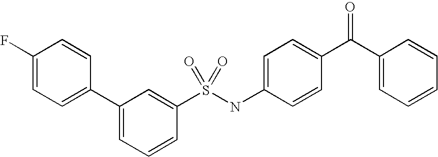 | 384 |
| 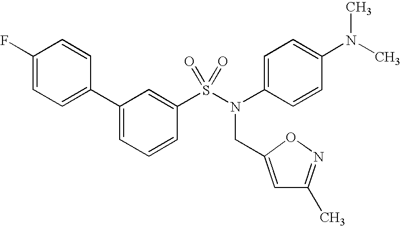 | 386 |
| 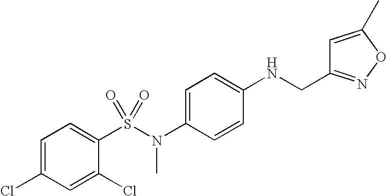 | 389 |
| 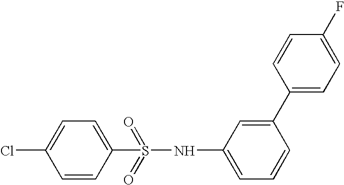 | 398 |
| 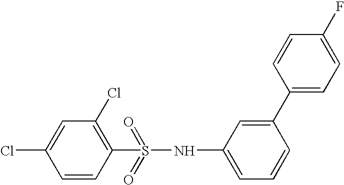 | 403 |
| 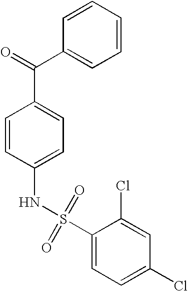 | 416 |
| 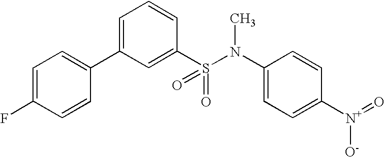 | 428 |
| 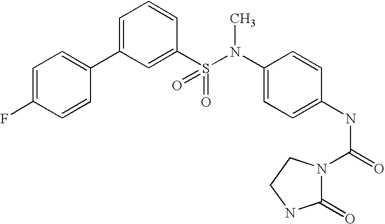 | 430 |
| 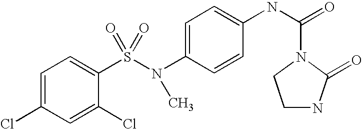 | 431 |
| 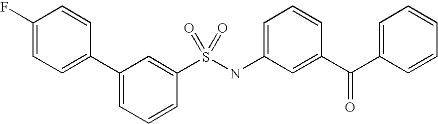 | 432 |
| 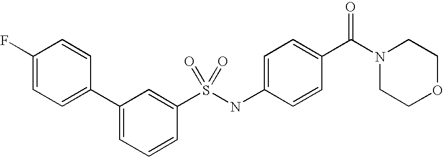 | 433 |
| 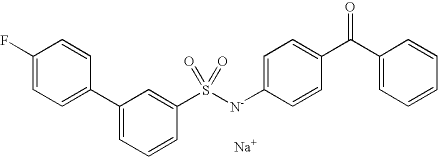 | 434 |
| 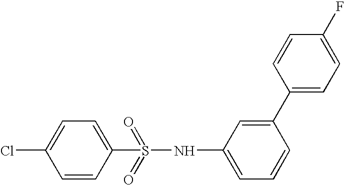 | 436 |
| 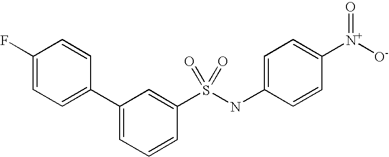 | 440 |
| 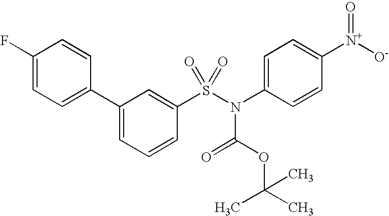 | 441 |
| 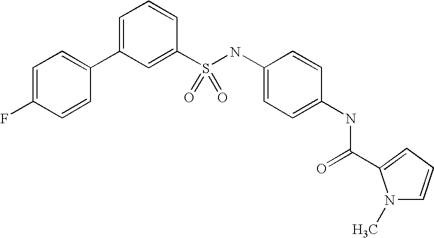 | 442 |
| 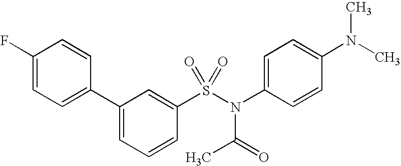 | 445 |
| 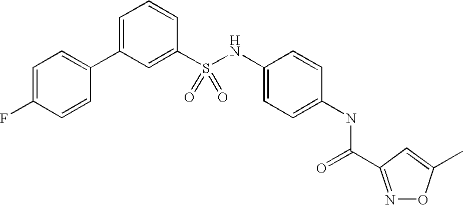 | 443 |
| 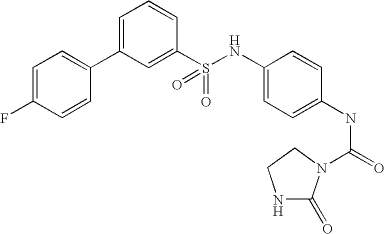 | 447 |
| 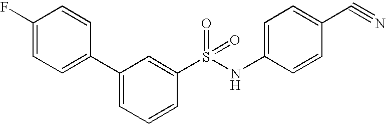 | 448 |
| 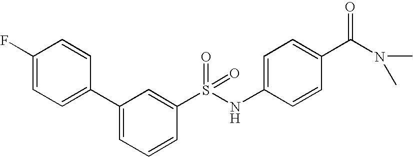 | 451 |
| 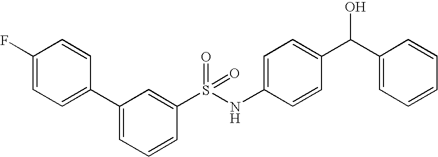 | 452 |
| 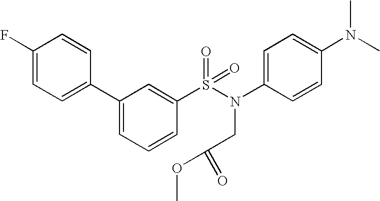 | 454 |
| 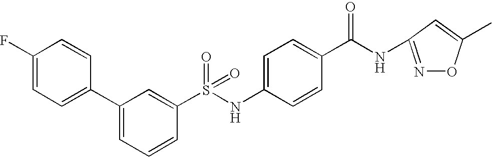 | 456 |
| 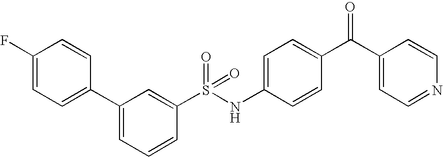 | 457 |
| 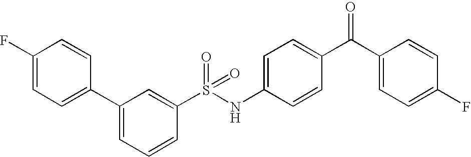 | 458 |

Specific examples of preferred compounds are

4′-fluoro-biphenyl-3-sulfonic acid (4-benzoyl-phenyl)-amide,

4′-fluoro-biphenyl-3-sulfonic acid (3-benzoyl-phenyl)-amide,

4′-fluoro-biphenyl-3-sulfonic acid (α-hydroxybenzyl-phenyl)-amide,

2-oxo-imidazolidine-1-carboxylic acid {4-[(4′-fluoro-biphenyl-3-sulfonyl)-methyl-amino]-phenyl}-amide.

Typical physiologically acceptable salts are e.g. acid addition salts (e.g. HCl, HBr, mesylate, etc.) and alkalimetal and alkaline earth metal salts (Na, K, Ca, Mg, etc.) conventionally used in the pharmaceutical field. Other suitable salts are e.g. ammonium, glucamine, amino acid etc. salts.

The compounds of formula (I) may be prepared by reacting a compound of formula (II)

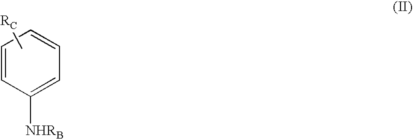

where R_{B} and R_{C} are as defined above, with a compound of formula (III)

R_{A}—SO₂hal (III)

where R_{A} is as defined above and hal is halogen.

The reaction may be carried out in conventional manner using methods well-known to the person skilled in the art.

The pharmaceutical compositions can contain one or more of the sulphonamides of the invention. The administration can be parenteral, subcutaneous, intravenous, intraarticular, intrathecal, intramuscular, intraperitoneal or intradermal injections, or intravenous infusion, or by transdermal, rectal, buccal, oromucosal, nasal, ocular routes or via inhalation or via implant. Alternatively or concurrently, administration can be by the oral route. The required dosage will depend upon the severity of the condition of the patient, for example, and such criteria as the patient's weight, sex, age, and medical history. The dose can also vary depending upon whether it is to be administered in a veterinary setting to an animal or to a human patient.

For the purposes of parenteral administration, compositions containing the sulphonamides of the invention are preferably dissolved in sterile water for injection and the pH preferably adjusted to about 6 to 8 and the solution is preferably adjusted to be isotonic. If the sulphonamide is to be provided in a lyophilized form, lactose or mannitol can be added as a bulking agent and, if necessary, buffers, salts, cryoprotectants and stabilizers can also be added to the composition to facilitate the lyophilization process, the solution is then filtered, introduced into vials and lyophilized.

Useful excipients for the compositions of the invention for parenteral administration also include sterile aqueous and non-aqueous solvents. The compounds of the invention may also be administered parenterally by using suspensions and emulsions as pharmaceutical forms. Examples of useful non-aqueous solvents include propylene glycol, polyethylene glycol, vegetable oil, fish oil, and injectable organic esters. Examples of aqueous carriers include water, water-alcohol solutions, emulsions or suspensions, including saline and buffered medical parenteral vehicles including sodium chloride solution, Ringers dextrose solution, dextrose plus sodium chloride solution, Ringers solution containing lactose, or fixed oils. Examples of solubilizers and co-solvents to improve the aqueous properties of the active compounds to form aqueous solutions to form parenteral pharmaceutical dosage forms are propylene glycol, polyethylene glycols and cyclodextrins. Examples of intravenous infusion vehicles include fluid and nutrient replenishers, electrolyte replenishers, such as those based upon Ringer's dextrose and the like.

Injectable preparations, such as solutions, suspensions or emulsions, may be formulated according to known art, using suitable dispersing or wetting agents and suspending agents, as needed. When the active compounds are in water-soluble form, for example, in the form of water soluble salts, the sterile injectable preparation may employ a non-toxic parenterally acceptable diluent or solvent as, for example, water for injection (USP). Among the other acceptable vehicles and solvents that may be employed are 5% dextrose solution, Ringer's solution and isotonic sodium chloride solution (as described in the Ph. Eur./USP). When the active compounds are in a non-water soluble form, sterile, appropriate lipophilic solvents or vehicles, such as fatty oil, for example, sesame oil, or synthetic fatty acid esters, for example, ethyl oleate or triglycerides, are used. Alternatively, aqueous injection suspensions which contain substances which increase the viscosity, for example, sodium carboxymethyl cellulose, sorbitol, and/or dextran, and optionally also contain stabilizers may be used.

Pharmaceutical preparations for oral (but systemic) administration can be obtained by combining the active compounds with solid excipients, optionally granulating a resulting mixture and processing the mixture or granules or solid mixture without granulating, after adding suitable auxiliaries, if desired or necessary, to give tablets or capsules after filling into hard capsules.

Suitable excipients are, in particular, fillers such as sugars, for example lactose or sucrose, mannitol or sorbitol, cellulose and/or starch preparations and/or calcium phosphates, for example tricalcium phosphate or calcium hydrogen phosphate, as well as binders, such as starches and their derivatives, pastes, using, for example, maize starch, wheat starch, rice starch, or potato starch, gelatine, tragacanth, methyl cellulose, hydroxypropylmethyl cellulose, sodium carboxymethyl cellulose, and/or polyvinyl pyrrolidone, derivatives, and/or, if desired, disintegrating agents, such as the above-mentioned starches, and also carboxymethyl-starch, cross-linked polyvinyl pyrrolidone, agar or alginic acid or a salt thereof, such as sodium alginate. Auxiliaries are, above all, flow-regulating agents and lubricants, for example, silica, talc, stearic acid or salts thereof, such as magnesium stearate or calcium stearate, with suitable coating, which if desired, are resistant to gastric juices and for this purpose, inter alia concentrated sugar solutions, which optionally contain gum arabic, talc, polyvinyl pyrrolidone, polyethylene glycol and/or titanium dioxide, lacquer solutions and suitable organic solvents or solvent mixtures, but also film coating using e.g. cellulose derivatives, polyethylene glycols and/or PVP derivatives may be used. In order to produce coatings resistant to gastric juices, solutions of suitable cellulose preparations such as acetyl cellulose phthalate or hydroxypropylmethyl cellulose phthalate, are used for coating. Dyestuffs or pigments may be added to the tablets or dragee coatings or to coatings for example, for identification or in order to characterize different combinations of active compound doses.

Solid dosage forms for oral administration include capsules, tablets, pills, troches, lozenges, powders and granules. In such solid dosage forms, the active compound may be admixed with at least one inert diluent such as sucrose, lactose or starch. Such dosage forms may also comprise, as is normal practice, pharmaceutical adjuvant substances, e.g., stearate lubricating agents or flavouring agents. Solid oral preparations can also be prepared with enteric or other coatings which modulate release of the active ingredients.

Liquid dosage forms for oral administration include pharmaceutically acceptable emulsions, solutions, suspensions, syrups and elixirs containing inert non-toxic diluents commonly used in the art, such as water and alcohol. Such compositions may also comprise adjuvants, such as wefting agents, buffers, emulsifying, suspending, sweetening and flavouring agents.

The compositions of the invention may also be administered by means of pumps, or in sustained-release form. The compounds of the invention may also be delivered to specific organs in high concentration by means of suitably inserted catheters, or by providing such molecules as a part of a chimeric molecule (or complex) which is designed to target specific organs.

Administration in a sustained-release form is more convenient for the patient when repeated injections for prolonged periods of time are indicated so as to maximize the comfort of the patient. Controlled release preparation can be achieved by the use of polymers to complex or adsorb the compounds of the invention. Controlled delivery can be achieved by selecting appropriate macromolecules (for example, polyesters, polyamino acids, polyvinyl pyrrolidone, ethylenevinylacetate, methylcellulose, carboxymethylcelluloase protamine zinc and protamine sulfate) as well as the method of incorporation in order to control release. Another possible method to control the duration of action by controlled release preparations is to incorporate the desired compounds into particles of a polymeric material such as polyesters, polyamino acids, hydrogels, poly (lactic acid) or ethylene vinylacetate copolymers. Alternatively, instead of incorporating the sulphonamide into these polymeric particles, the sulphonamide can be entrapped into microparticles, prepared, for example, by coacervation techniques or by interfacial polymerization, for example, hydroxymethylcellulose or gelatin-microcapsules and poly (methylmethacrylate) microcapsules, respectively, or in colloidal drug delivery systems, for example liposomes, albumin microspheres, microemulsions, nanoparticles, and nano-capsules or in macroemulsions. The above-mentioned technique may be applied to both parenteral and oral administration of the pharmaceutical formulation.

The pharmaceutical compositions of the present invention can be manufactured in a manner which is in itself know, for example, by means of conventional mixing, granulating, dragee-making, dissolving, lyophilizing or similar processes.

The compounds of the invention are potent collagen receptor inhibitors and useful for inhibiting or preventing the adhesion of cells on collagen or the migration and invasion of cells through collagen, in vivo or in vito. The now described compounds inhibit the migration of malignant cells and are thus for treating diseases such as cancers, including prostate, and melanoma, especially where α2β1 integrin dependent cell adhesion/invasion/migration may contribute to the malignant mechanism.

The compounds of the invention also inhibit adhesion of platelets to collagen and collagen-induced platelet aggregation. Thus, the compounds of the invention are useful for treating patients in need of preventative or ameliorative treatment for thromboembolic conditions i.e diseases that are characterized by a need to prevent adhesion of platelets to collagen and collagen-induced platelet aggregation, for example treatment and prevention of stroke, myocardial infraction unstable angina pectoris diabetic rethinopathy or retinal vein occlusion.

Pharmacological tests
A Cell Invasion Assay was Used to Demonstrate the Anti-Cancer Potential of the Inhibitors in vitro

The ability to interact with extracellular matrix basement membranes is essential for the malignant cancer cell phenotype and cancer spread. α2β1 levels are known to be upregulated in tumorigenic cells. The overexpression regulates cell adhesion and migration to and invasion through the extracellular matrix. By blocking the interaction between extracellular matrix components like collagen and α2β1 it is possible to block cancer cell migration and invasion in vitro. Prostate cancer cells (PC-3) expressing α2β1 endogenously were used to test the in vitro anticancer potential of the inhibitors of the present invention.

### Experimental Procedure

Invasion of PC-3 cells (CRL-1435, ATCC) through Matrigel was studied using BD Biocoat invasion inserts (BD Biosciences). Inserts were stored at −20° C. Before the experiments inserts were allowed to adjust to the room temperature. 500 μl of serum free media (Ham's F12K medium, 2 mM L-glutamine, 1.5 g/l sodium bicarbonate) was added into the inserts and allowed to rehydrate at 37° C. in cell incubator for two hours. The remaining media was aspirated. PC-3 cells were detached, pelleted and suspended into serum free media (50000 cells/500 μl). 300 μl of cell suspension was added into the insert in the absence (control) or presence of the inhibitor according to the present invention. Inserts were placed on the 24-well plates; each well containing 700 μl of cell culture media with 3% of fetal bovine serum as chemo-attractant. Cells were allowed to invade for 72 hours at 37° C. in cell incubator. Inserts were washed with 700 μl PBS, and fixed with 4% paraformaldehyde for 10 minutes. Paraformaldehyde was aspirated and cells were washed with 700 μl of PBS and inserts were stained by incubation with hematoxylin for 1 minute. The stain was removed by washing the inserts with 700 μl of PBS. Inserts were allowed to dry. Fixed invaded cells were calculated under the microscope. Invasion % was calculated as a comparison to the control.

Cell invasion assay is used as an in vitro cancer metastatis model. The sulfonamide molecules have been shown to inhibit tumor cell invasion in vitro (Table 2). Some structures inhibit invasion even with submicromolar concentrations.

### A Platelet Function Analyzer PFA-100 was Used to Demonstrate the Anti-Thrombotic Potential of the α2β1 Inhibitors

A platelet function analyzer PFA-100 was used to demonstrate the possible antithrombotic effects of α2β1 modulators. The PFA-100 is a high shear-inducing device that simulates primary haemostasis after injury of a small vessel. The system comprises a test-cartridge containing a biologically active membrane coated with collagen plus Epinephrin. An anticoaculated whole blood sample was run through a capillary under a constant vacuum. The platelet agonist (Epinephrin) on the membrane and the high shear rate resulted in activation of platelet aggregation, leading to occlusion of the aperture with a stable platelet plug. The time required to obtain full occlusion of the aperture was designated as the “closure time”. Each compound was added to the whole blood sample and the closure time was measured with PFA-100. If the closure time was increased when compared to the control sample the compound was suggested to have antithrombotic activity.

### Experimental Procedure

Blood was collected from a donor via venipuncture into evacuated blood collection tubes containing 3.2% buffered sodium sitrate as anticoagulant. Blood was aliquoted into 15 mL tubes and treated with either inhibitory compounds or controls (DMSO). Samples were kept at room temperature with rotation for 10 minutes and after that the closure time of the blood was measured.

Acquisitions resulting in a closure time exceeding the range of measurement of the instrument (>300 seconds) were assigned a value of 300 seconds. Mean and standard deviations were calculated for each treatment. Student's t-test was applied to the resultant data.

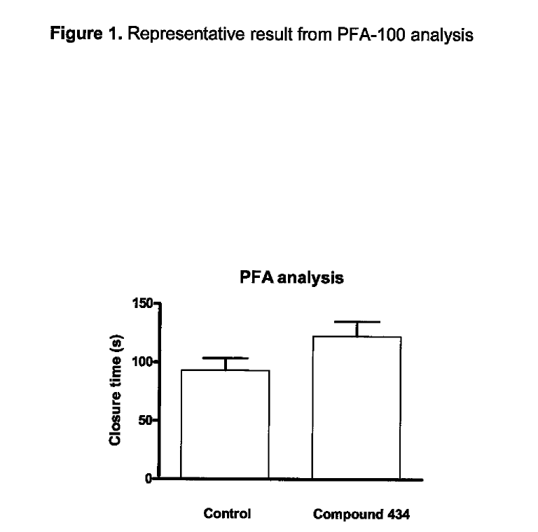

The compound 434 was shown to increase the closure time of the blood (FIG. 1.).

TABLE 2

| Compound | EC50 in cell |
|---|---|
| number | invasion (μM) |
| 384 | 0.8 |
| 430 | 12 |
| 432 | 1 |
| 434 | 0.8 |
| (salt of 384) | |
| 440 | 8.3 |
| 448 | 27 |
| 452 | 0.8 |

The test results showed that the compounds of the present invention have an anti-cancer and antithrombotic activity in vitro.

### Adhesion Assay Method

Chinese Hamster Ovary (CHO) cell clone expressing wild type α2 integrin was used in cell adhesion assay. Cells were suspended in serum free medium containing 0.1 mg/ml cycloheximide (Sigma) and the compounds were preincubated with the cells prior to transfer to the wells. Cells (150000/well) were allowed to attach on collagen type I coated wells (in the presence and absence of inhibitor compounds) for 2 h at +37° C. and after that non-adherent cells were removed. Fresh serum free medium was added and the living cells were detected using a cell viability kit (Roche) according to the manufacturer's protocol.

TABLE 3
The effect of integrin inhibitors on CHO-α2β1
adhesion on type I collagen

| Compound | Inhibition % at |
|---|---|
| number | 50 uM |
| 353 | 11 |
| 354 | 78 |
| 355 | 18 |
| 358 | 71 |
| 359 | 42 |
| 378 | 22 |
| 383 | 26 |
| 384 | 65 |
| 403 | 56 |
| 430 | 53 |
| 432 | 72 |
| 434 | 78 |
| 437 | 36 |
| 440 | 59 |
| 442 | 21 |
| 448 | 58 |
| 452 | 83 |
| 456 | 13 |
| 458 | 66 |

The following examples illustrate the invention but are not intended to limitate the scope of the invention.

### General Procedures

### Sulfonyl Chloride Coupling Procedure 1: Coupling of Sulfonyl Chloride to Amine in Acetonitrile

To a stirred solution of the amine (0.75 mmol) and triethylamine (0.75 mmol) in anhydrous acetonitrile (1 ml) at 0° C. was added 2,4-dichloro-benzenesulphonyl chloride (0.50 mmol) in acetonitrile (1 ml). The mixture was stirred at this temperature for 2-3 hours and/or warmed up to ambient temperature and stirred until reaction had completed by TLC.

The solvent was removed in vacuo and the residue partitioned between ethyl acetate (25 ml) and saturated aqueous sodium bicarbonate solution (25 ml). The organic layer was separated and further washed with sodium bicarbonate (2×25 ml), brine (2×25 ml), dried over sodium sulphate and concentrated down. The product was purified either by flash chromatography (cyclohexane/ethyl acetate eluent on silica), preparative HPLC (acetonitrile/water on C18 silica column), using a silica cartridge (cyclohexane/ethyl acetate eluent on silica), preparative HPLC (either reverse C18 or normal silica) or by recrystalisation from methanol.

### Sulfonyl Chloride Coupling Procedure 2: Coupling of Sulfonyl Chloride to Amine in Pyridine

To the aniline (0.6 mmol) in pyridine (5 ml) stirring at 0° C. was added sulfonyl chloride (1 equivalent) in pyridine (5 ml) and the reaction was allowed to warm to room temperature overnight. The solvent was evaporated and the resulting residue taken up in EtOAc and washed with aqueous solution of base. The rest of the workup as was for sulfonyl chloride procedure 1.

### Sulfonyl Chloride Coupling Procedure 3: Coupling of Sulfonyl Chloride to Amine in Tetrahydrofuran

To a solution of 4-(dimethylamino)benzylamine dihydrochloride and potassium carbonate in THF (anhydrous, 3 ml) was added 3-bromobenzene-1-sulfonyl chloride drop wise in THF (2 ml) with cooling and stirring, it was noted that some material was insoluble at the intended concentration, further THF (15 ml) and CH₃CN (5 ml) were added. The reaction was allowed to warm to room temperature overnight with stirring. The solvent was evaporated and the resulting residue partitioned between CH₂Cl₂ and H₂O. The aqueous layer was washed with further CH₂Cl₂, the organic portions combined, and purified by flash silica column chromatography [cyclohexane/EtOAc (8:2-7:3)].

### Suzuki Coupling Procedure 1

To a degassed mixture of toluene (4 ml) and 2M aqueous Na₂CO₃ (2 ml) was added the bromosulfonamide (0.26 mmol), the phenyl boronic acid (0.28 mmol) and tetrakis (triphenylphosphine) palladium(0) (3 to 5 mol %). The mixture was refluxed for 48 hours. The reaction was cooled, filtered through celite and the celite cake washed with AcOEt (3*50 ml). The organic layer was dried and residue purified.

### Suzuki Coupling Procedure 2

To a degassed solution of 3-bromo-N-[4-(dimethylamino)phenyl]-benzenesulfonamide (100 mg, 0.28 mmol) in toluene (2.5 ml) was added tetrakis (triphenylphosphine) palladium(0) (10 mg, 3 mol %), pyridyl boronic acid (38 mg, 0.28 mmol) in ethanol (1 ml) and sodium carbonate (150 mg, 1.41 mmol) in water (1 ml). The reaction was refluxed for 48 hours. The workup procedure was for Suzuki coupling procedure 1.

### Methylation Procedure 1

To a solution of the indole (1 eqv) in N,N-dimethylformamide solvent (0.7 ml/mmol) was added anhydrous potassium carbonate (0.20 eqv.) and dimethyl carbonate (2.1 eqv.). The mixture was stirred under reflux for 2-3 hours before being left to stir at room temperature overnight. The mixture was cooled (5° C.) and ice-cold water (1.5 ml/mmol) was added slowly. The precipitated product is filtered under suction, washed with water and dried in vacuo to give the corresponding N-methylated indole which was then purified.

### Methylation Procedure 2

The sulfonamide (0.14 mmol) was stirred at 0° C. in DMF (anhydrous, 10 ml) with sodium hydride (1 equivalent) for 30 mins. Methyl iodide (1 equivalent) was added and the reaction allowed to rise to room temperature with stirring. The reaction was monitored by TLC and if necessary further methyl iodide added. The reaction solution was then diluted into distilled water and extracted with ethyl acetate, the ethyl acetate was repeatedly washed with distilled water and then brine before being dried (sodium sulphate) and evaporated to dryness prior to purification.

### Methylation Procedure 3

The sulphonamide (1 eqv) and 1,4-diazabicyclo[2.2.2]octane (0.2 eqv) were heated in DMF/Dimethyl carbonate (1/10 mixture, 10 ml) at 95° C. for 1 to 3 days. The mixture was allowed to cool to room temperature and partitioned between ethyl acetate (15 ml) and water (15 ml). The organic layer was separated and washed with water (10 ml), 10% citric acid (2×10 ml) and again with water (2×10 ml). The organics were dried over sodium sulphate and concentrated in vacuo.

### EXAMPLE 1

### Compound 384 4′-fluoro-biphenyl-3-sulfonic acid (4-benzoyl-phenyl)-amide

Reaction was carried out according to procedure 2 for sulfonyl chloride coupling.

A yellow solid was recovered: 13 mg (17%).

¹H NMR (300 MHz, CDCl₃ δ8.00-7.99 (t, 1H, J=1.8 Hz), 7.84-7.69 (m, 6H), 7.60-7.43 (m, 6H), 7.26-7.14 (m, 4H), 7.02 (s, 1H)

LCMS Rₜ 15.4 min.; purity 96%; MS m/z no ionisation.

### EXAMPLE 2

### Compound 434 4′-fluoro-biphenyl-3-sulfonic acid (4-benzoyl-phenyl)-amide sodium salt

To a solution of Compound 384 (139 mg, 0.32 mmol) in MeOH at room temperature 0.5 M sodium methoxide in methanol (0.68 ml, 0.34 mmol) was added. The reaction mixture slowly goes yellow in colour and was stirred for a further 48 hours. After which time the solvent was removed under reduced pressure to yield a yellow gum. This was triturated with t-butylmethyl-ether, the ether layer decanted and the resultant residue evaporated to dryness to give a cream coloured solid (97 mg, 67%).

¹H NMR (400 MHz, DMSO) δ 7.94 (t, 1H, J=1.8 Hz), 7.72 (dt, 1H, J =1.4, 7.8), 7.66 (2H, dd, J=5.3, 8.8), 7.66-7.62 (m, 1H), 7.54 (d, 2H, J=8.1), 7.55-7.51 (m, 1H), 7.49-7.43 (m, 3H), 7.39 (d, 2H, J=8.8), 7.30 (t, 2H, J=8.8), 6.84 (d, 2H, J=8.8).

LCMS Rₜ 15.7 min.; purity 96.2%; MS m/z 432 [M+H]⁺.

(C₂₅H₁₇NO₃SFNa Required: C 66.2, H 3.8, N 3.1, Na 5.1; Found C 64.2, H 3.62, N 3.0, Na 5.9).

### EXAMPLE 3

### Compound 430 2-oxo-imidazolidine-1-carboxylic acid {4-[(4′-fluoro-bi-phenyl-3-sulfonyl)-methyl-amino]-phenyl)-amide

4′-fluoro-biphenyl-3-sulfonic acid (4-amino-phenylyamide was stirred in acetonitrile (anhydrous, 10 ml) with pyridine (2 equivalents) and 2-oxo-1-imidazolidinecarbonyl chloride (1 equivalent) at room temperature for 2 hours. Heating (90° C.) was necessary, followed by addition of further portions of pyridine and acid chloride and heating (95° C.) for 4 hours.

Reaction cooled and solvent evaporated. The residue was dissolved in ethyl acetate/water (1:1), the ethyl acetate collected and the water washed with ethyl acetate. The organic washes were combined and washed with water and brine, dried with magnesium sulfate and evaporated.

¹H NMR (300 MHz, CDCl₃) δ 8.92-8.90 (d, 2H), 8.50-8.45 (t, 1H,), 8.02-7.98 (t, 2H,) 6.6 Hz), 7.71-7.26 (m, 5H), 7.13-7.07 (t, 1H), 7.03-7.00 (d, 1H, J=8.6 Hz), 4.04-3.98 (t, 1H), 3.57-3.51 (t, 2H), 3.15 (s, 2H), 1.98 (s, 2H)

LCMS Rₜ 13.5 min.; purity 96%; MS m/z 469.5, [M+H]⁺.

### EXAMPLE 4

### Compound 432 4′-fluoro-biphenyl-3-sulfonic acid (3-benzoyl-phenyl)-amide

4-fluoro-biphenyl-3-sulfonic acid (3-benzoyl-phenyl)-amide was synthesized from the respective amine and sulfonyl chloride using the sulfonyl chloride procedure 3. Purification after an aqueous workup was achieved by preparative HPLC.

¹H NMR (300 MHz, CDCl₃) δ 7.50-7.09 (m, 15H), 6.80 (m, 2H).

LCMS Rₜ 15.50 min.; purity 87.9%; MS m/z 432.5, [M+H]⁺.

In the same way as described in Examples 1-4, but using appropriate starting compounds, the following compounds of the invention were synthesized (the compound numbers refer to those used in Table 1):

### Compound 353 4′-fluoro-biphenyl-3-sulfonic acid (4-dimethylamino-phenyl)-(2-methoxy-ethyl)-amide

¹H NMR (300 MHz, CDCl₃) δ 7.77 (d, 1H, J=2.0 Hz), 7.73-7.71 (d, 1H, J=7.8 Hz), 7.62-7.65 (d, 1H, J=7.7 Hz), 7.54-7.46 (m, 3H), 7.15-7.09 (t, 2H, J=8.7 Hz), 6.92-6.89 (dd, 2H, J=2.0 Hz, 8.8 Hz), 6.61-6.58 (d, 2H, J=8.8 Hz), 3.74-3.69 (t, 2H, J=6.4 Hz, 6.1 Hz), 3.47-3.43 (t, 2H, J=6.1 Hz, 6.4 Hz), 3.28-3.27 (d, 3H, J=2.0 Hz), 3.09-2.95 (d, 6H, J=2.0 Hz)

LCMS Rₜ 16.1 min.; purity 98%; MS m/z 429.4, [M+H]⁺.

### Compound 354 4′-fluoro-biphenyl-3-sulfonic acid (2-dimethylamino-ethyl)-(4-dimethylamino-phenyl)-amide

¹H NMR (300 MHz, CDCl₃) δ 7.74-7.70 (m, 2H), 7.63-7.61 (d, 1H, J=7.8 Hz), 7.54-7.48 (m, 3H), 7.15-7.09 (t, 2H, J=8.5 Hz), 6.91-6.88 (d, 2H, J =8.8 Hz), 6.61-6.58 (d, 2H, J=8.8 Hz), 3.65-3.60 (t, 2H, J=7.4 Hz), 2.95 (s, 6H), 2.44-2.40 (t, 2H, J=7.2 Hz), 2.22-2.21 (d, 6H)

LCMS Rₜ 18.3 min.; purity 95%; MS m/z 442.4 [M+H]⁺.

### Compound 355 4′-fluoro-biphenyl-3-sulfonic acid (4-dimethylamino-phenyl)-(2-morpholin-4-yl-ethyl)-amide

¹H NMR (300 MHz, CDCl₃) δ 7.72 (s, 2H), 7.65-7.62 (dd, 1H, J=2.0 Hz, 7.9 Hz), 7.52-7.45 (m, 3H), 7.15-7.09 (t, 2H, J=8.7 Hz), 6.92-6.89 (d, 2H, J=9.2 Hz), 6.61-6.58 (d, 2H, J=9.1 Hz), 3.65-3.63 (m, 6H), 2.96-2.95 (d, 6H, J=2.0 Hz), 2.49-2.42 (m, 6H)

LCMS Rₜ 15.4 min.; purity 98%; MS m/z 484.3, [M+H]⁺.

### Compound 358 4′-fluoro-biphenyl-3-sulfonic acid (4-dimethylamino-phenyl)-(2-imidazol-1-yl-ethyl)-amide

¹H NMR (300 MHz, CDCl₃) δ 7.74-7.72 (m, 1H), 7.65 (s, 1H), 7.53-7.42 (m, 5H), 7.14-7.09 (m, 2H), 7.04 (s, 1H), 6.93 (s, 1H), 6.80-6.77 (d, 2H, J=7.3 Hz), 6.59-6.56 (d, 2H, J=8.2 Hz), 4.14-4.10 (t, 2H, J=6.4 Hz), 3.87-3.83 (t, 2H, J=6.4 Hz), 2.96 (s, 6H)

LCMS Rₜ 14.3 min.; purity 96%; MS m/z 465.4, [M]⁺.

### Compound 359 4′-fluoro-biphenyl-3-sulfonic acid (4-dimethylamino-phenyl)-(2-hydroxy-ethyl)-amide

¹H NMR (300 MHz, CDCl₃) δ 7.76-7.73 (d, 2H, J=7.7 Hz), 7.69-7.68 (d, 2H, J=11.6 Hz), 7.62-7.46 (m, 3H), 7.16-7.10 (t, 2H, J=8.4 Hz), 6.94-6.91 (d, 2H, J=8.7 Hz), 6.64-6.62 (d, 2H, J=7.8 Hz), 3.68 (s, 4H), 2.96 (s, 6H)

LCMS Rₜ 14.3 min.; purity 98%; MS m/z 415.4, [M+H]⁺.

### Compound 378 4′-fluoro-biphenyl-3-sulfonic acid (4-(1,3-oxazol-5-yl)-phenyl)-amide

¹H NMR (300 MHz, CDCl₃) δ 7.92 (d, 2H), 7.72 (m, 2H), 7.56 (d, 2H, J=8.7 Hz), 7.45 (m, 2H(, 7.29 (m, 2H), 7.15 (m, 3h), 6.57 (s, 1H).

LCMS Rₜ 13.68 min.; purity 97.8%; MS m/z no ionisation.

### Compound 383 4′-fluoro-biphenyl-3-sulfonic acid (4-acetyl-phenyl)-amide

¹H NMR (300 MHz, CDCl₃) δ 8.02-8.01 (t, 1H, J=1.8 Hz), 7.88-7.85 (m, 2H), 7.82-7.78 (m, 1H), 7.73-7.70 (m, 1H), 7.55-7.44 (m, 3H), 7.34 (s, 1H), 7.56-7.10 (m, 4H), 2.53 (s, 3H)

LCMS Rₜ 13.6 min.; purity 96%; MS m/z no ionisation.

### Compound 386 2,4-dichloro-N-(1,2-dimethyl-1H-indol-5-yl)-N-(3-methyl-isoxazol-5-ylmethyl)-benzenesulfonamide

¹H NMR (300 MHz, CDCl₃) δ 7.76-7.73 (m, 2H), 7.65-7.45 (m, 4H), 7.15-7.10 (t, 2H, J=8.7 Hz), 6.86-6.83 (dd, 2H, J=2.2 Hz, 7.0 Hz), 6.54-6.51 (d, 2H, J=9.0 Hz), 6.14 (s, 1H), 4.73 (s, 2H), 2.92 (s, 6H), 2.35 (s, 3H)

LCMS Rₜ 16.0 min.; purity 91%; MS m/z 466.5, [M+H]⁺.

### Compound 389 2,4-dichloro-N-methyl-N-(4-[(5-methyl-isoxazol-3-ylmethyl)-amino]-phenyl}-benzenesulfonamide

¹H NMR (300 MHz, CDCl₃) δ 7.76-7.73 (d, 1H, J=8.5 Hz), 7.52-7.51 (d, 1H, J=2.1 Hz), 7.26-7.21 (m, 1H), 6.98-6.95 (d, 2H, J=8.8 Hz), 6.56-6.53 (d, 2H, J=8.8 Hz), 5.94 (s, 1H), 4.31 (s, 2H), 3.36 (s, 3H), 2.39 (s, 3H)

LCMS Rₜ 14.7 min.; purity 96%; MS m/z 426.4, [M]⁺.

### Compound 398 4′-fluoro-biphenyl-3-sulfonic acid (4-chlorophenyl)-amide

¹H NMR (400 MHz, CDCl₃) δ 7.75 (d, 2H, J=7.6 Hz), 7.45-7.38 (m, 4 H), 7.31-7.26 (m, 4H), 7.12-7.08 (m, 3H).

LCMS Rₜ 15.62 min.; purity 97.8%; no ionization.

### Compound 403 2,4-cichlorophenylsulfonic acid (3-(4-fluorophenyl)phenyl)-amide

¹H NMR (300 MHz, CDCl₃) δ 7.95 (d, 1H, J=8.8 Hz), 7.51 (d, 1H, J=2.0 Hz), 7.43 (m, 2H), 7.33-7.26 (m, 4H), 7.13-7.08 (m, 3H).

LCMS Rₜ 16.21 min.; purity 95.39%; MS m/z 436.6, [M+CH₃CN]⁺.

### Compound 416 2,4-dichlorophenyl-sulfonic acid (4-benzoyl-phenyl)-amide

¹H NMR (400 MHz, CDCl₃) δ 8.05 (m, 2H), 7.70 (m, 4 H), 7.58 (t, 1H), 7.47-7.36 (m, 4H), 7.20 (d, 2H).

LCMS Rₜ 14.64 min.; purity 91.44%; MS m/z no ionisation.

### Compound 428 Intermediate: 4′-fluoro-biphenyl-3-sulfonic acid (4-nitro-phenyl)-amide

¹H NMR (300 MHz, CDCl₃) δ 8.15-8.06 (m, 3H), 7.86-7.74 (m, 2H), 7.59-7.46 (m, 4H), 7.27-7.24 (dd, 2H, J=2.3 Hz, 6.9 Hz), 7.17-7.11 (t, 2H, J=8.6 Hz).

LCMS Rₜ 13.6 min.; purity 99%; MS m/z no ionization.

### Compound 428 4′-fluoro-biphenyl-3-sulfonic acid methyl-(4-nitro-phenyl)-amide

¹H NMR (300 MHz, CDCl₃) δ 8.21-8.18 (d, 2H, J=9.3 Hz), 7.76-7.72 (m, 2H), 7.54-7.35 (m, 6H), 7.17-7.11 (t, 2H, J=8.7 Hz), 3.27 (s, 3H)

LCMS Rₜ 15.7 min.; purity 96%; MS m/z no ionisation.

### Compound 431 2-oxo-imidazolidine-1-carboxylic acid {4-[(2,4-dichloro-benzenesulfonyl)-methyl-amino]-phenyl}-amide

¹H NMR (300 MHz, CDCl₃) δ 7.77-7.74 (d, 1H, J=8.6 Hz), 7.51-7.40 (d, 1H, J=2.3 Hz), 7.44-7.41 (d, 2H, J=8.8 Hz), 7.26-7.22 (dd, 1H, J=3.2 Hz, 9.6 Hz, 8.6 Hz, 2.2 Hz), 7.14-7.11 (d, 2H, J=8.8 Hz), 5.11 (bs, 1H), 4.07-4.01 (t, 2H, J=7.7 Hz, 8.6 Hz), 3.58-3.52 (t, 2H, J=8.3 Hz, 7.9 Hz), 3.40 (s, 3H)

LCMS Rₜ 13.0 min.; purity 99%; MS m/z 443.4, [M]⁺.

### Compound 433 4′-fluoro-biphenyl-3-sulfonic acid (4-(N-morpholinocarbonyl)-phenyl)-amide

¹H NMR (300 MHz, CDCl₃) δ

LCMS Rₜ 12.57 min.; purity 91.59%; MS m/z 441.6, [M]⁺.

### Compound 436 4′-fluoro-biphenyl-3-sulfonic acid (4-chlorophenyl)-N-methylamide

¹H NMR (300 MHz, CDCl₃) δ 7.54-7.42 (m, 9H), 7.12 (t, 2H, J=9.0 Hz), 7.01 (dd, 1H), 3.21 (s, 3H).

LCMS Rₜ min.; purity %; MS m/z.

### Compound 440 4′-fluoro-biphenyl-3-sulfonic acid (4-nitro-phenyl)-amide

¹H NMR (300 MHz, CDCl₃) δ 8.15-8.11 (dd, 2H, J=2.1 Hz, 7.0 Hz), 8.07-8.06 (t, 1H, J=1.9 Hz), 7.86-7.74 (m, 3H), 7.58-7.56 (d, 1H, J=7.7 Hz), 7.50-7.46 (m, 2H), 7.27-7.24 (dd, 2H, J=3.1 Hz, 7.0 Hz, 6.1 Hz, 2.1 Hz), 7.17-7.08 (t, 2H, J=8.6 Hz)

LCMS Rₜ 14.7 min.; purity 96%; MS m/z no ionisation.

### Compound 441 4′-fluoro-biphenyl-3-sulfonic acid tert-butyl carbamate-(4-nitro-phenyl)-amide

¹H NMR (300 MHz, CDCl₃) δ 8.32-8.29 (m 2H), 8.16-8.15 (m, 1H), 7.93-7.85 (m, 2H), 7.68-7.55 (m, 4), 7.47-7.44 (dd, 3H, J=2.1 Hz, 7.0 Hz), 7.22-7.16 (t, 2H, J=8.6 Hz), 1.35 (s, 10H)

LCMS Rₜ 16.8 min.; purity 92%; MS m/z no ionisation.

### Compound 442 1-methyl-1H-pyrrole-2-carboxylic acid [4-(4′-fluoro-bi-phenyl-3-sulfonylamino)-phenyl]-amide

¹H NMR (300 MHz, CDCl₃/d₄-MeOH) δ 7.52-7.51 (m, 1H), 7.35-7.26 (m, 2H), 6.98-6.90 (m, 6H), 6.67-6.61 (m, 2H), 5.95-5.93 (m, 1H), 3.75 (s, 3H)

LCMS Rₜ 13.6/13.7 min.; purity 100%; MS m/z 450.5, [M+H]⁺.

### Compound 443 5-methyl-isoxazole-3-carboxylic acid [4-(4′-fluoro-biphenyl-3-sulfonylamino)-phenyl]-amide

¹H NMR (300 MHz, CDCl₃/d₄-MeOH) δ 7.84-7.83 (m, 1H), 7.64-7.62 (m, 2H), 7.51-7.39 (m, 5H), 7.10-7.04 (m, 4H), 6.46 (s, 1H), 2.45 (s, 3H)

LCMS Rₜ 14.1 min.; purity 99%; MS m/z 542.4, [M+H]⁺.

### Compound 445 4′-fluoro-biphenyl-3-sulfonic acid acetyl-(4-dimethylamino-phenyl)-amide

¹H NMR (400 MHz, CDCl₃) δ 8.27 (t, 1H, J=1.8), 8.01 (d, 1H, J=7.8), 7.83 (d, 1H, J=7.8), 7.65-7.59 (m, 3H), 7.18 (t, 2H, J=8.6), 7.31 (d, 2H, J=8.9), 6.74 (d, 2H, d, J=8.9), 3.05 (s, 6H), 1.92 (s, 3H).

LCMS Rₜ 17.6 min.; purity 94:1%; MS m/z 413 [M+H]⁺.

### Compound 447 2-oxo-imidazolidine-1-carboxylic acid [4-(4′-fluoro-biphenyl-3-sulfonylamino)-phenyl]-amide

¹H NMR (300 MHz, DMSO) δ 7.91-7.82 (m, 2H), 7.68-7.60 (m, 4H), 7.35-7.29 (m, 4H), 7.04-7.01 (d, 2h, J=8.9 Hz), 3.81-3.76 (t, 2H, J=7.5 Hz, 9.0 Hz).

LCMS Rₜ 12.5 min.; purity 98%; MS m/z 455.4, [M+H]⁺.

### Compound 448 4′-fluoro-biphenyl-3-sulfonic acid (4-cyano-phenyl)-amide

¹H NMR (300 MHz, CDCl₃) δ 8.02 (s, 1H), 7.82-7.73 (m, 2H), 7.58-7.75 (m, 5H), 7.26-7.12 (m, 4H).

LCMS Rₜ 14.3 min.; purity 98%; MS m/z 351.4, [M−H]⁻.

### Compound 451 (4′-fluoro-biphenyl)-3-sulfonylamino)-N,N-dimethyl-benzamide

¹H NMR (400 MHz, CDCl₃) δ 7.95 (t, 1H, J=1.7 Hz), 7.77 (d, 1H, J=8.1 Hz), 7.72 (d, 1H, J=8.1 Hz), 7.53 (t, 1H, J=7.8 Hz), 7.47 (dd, 2H, J=5.2, 3.5 Hz), 7.34 (d, 2H, J=8.3 Hz), 7.16 (m, 4H), 7.07 (brs, 1H), 3.09 (brs, 3H), 2.93 (brs, 3H).

LCMS Rₜ 12.65 min.; purity 93.6%; MS m/z 399.4 [M+H]⁺.

### Compound 452 4′-fluoro-biphenyl-3-sulfonic acid (4-benzoyl-phenyl)-amide

¹H NMR (400 MHz, CDCl₃) δ 7.89 (t, 1H, J=1.8 Hz), 7.74 (d, 1H, J=7.9 Hz), 7.71 (d, 1H, J=7.9 Hz), 7.51 (t, 1H, J=8.0 Hz), 7.44 (d, 1H, J=5.2 Hz), 7.43 (d, 1H, J=5.2 Hz), 7.31 (m, 7H), 7.11 (m, 4H), 6.64 (brs, 1H), 5.58 (d, 1H, J=3.3 Hz), 2.21 (d, 1H, J=3.5 Hz).

LCMS Rₜ 13.36 min.; purity 92.9%; MS m/z 416.4 [M+H—OH]⁺.

### Compound 454 [(4-dimethylamino-phenyl)-(4′-fluoro-biphenyl-3-sulfonyl)-amino]-acetic acid methyl ester

¹H NMR (400 MHz, CDCl₃) δ 7.83 (t, 1H, J=1.8), 7.75 (ddd, 1H, J=1.2, 1.8, 7.8), 7.70 (ddd, 1H, J=1.2, 1.8, 7.7), 7.54 (t, 1H, J=7.8), 7.51 (dd, 2H, J=5.6, 8.8), 7.15 (t, 2H, J=8.8), 7.07 (d, 2H, J=8.8), 6.60 (d, 2H, J=8.8), 4.43 (s, 2H), 3.71 (s, 3H), 2.96 (s, 6H).

LCMS Rₜ 16.8 min.; purity 91.4%; MS m/z 443 [M+H]⁺.

### Compound 456 4-(4′-fluoro-biphenyl-3-sulfonylamino)-N-(5-methyl-isoxazol-3-yl)-benzamide

¹H NMR (400 MHz, d-DMSO) 11.12 (brs, 1H), 10.86 (brs, 1H), 8.05 (s, 1H), 7.89 (m, 3H), 7.80 (d, 1H, J=7.1 Hz), 7.69 (m, 3H), 7.35 (t, 2H, J=7.1 Hz), 7.23 (d, 2H, J=8.1 Hz), 6.69 (s, 1H), 2.38 (s, 3H).

LGMS Rₜ 13.83 min.; purity 94.7%; MS m/z 452.4 [M+H]⁺.

### Compound 457 4′-fluoro-biphenyl-3-sulfonic acid [4-(pyridine-4-carbonyl)-phenyl]-amide

¹H NMR (400 MHz, d-DMSO) 11.08 (brs, 1H), 8.76 (brd, 2H, J=4.1 Hz), 8.08 (brt, 1H, J=1.7 Hz), 7.94 (d, 1H, J=7.8 Hz), 7.84 (d, 1H, J=7.8 Hz), 7.70 (m, 5H), 7.52 (d, 2H, J=5.8 Hz), 7.34 (m, 4H).

LCMS Rₜ 13.56 min.; purity 89.4%; MS m/z 433.4 [M+H]⁺.

### Compound 458 4′-fluoro-biphenyl-3-sulfonic acid [4-((4-fluorophenyl)-4-carbonyl)-phenyl]-amide

¹H NMR (400 MHz, CDCl₃) δ 8.04 (t, 1H, J=1.7 Hz), 7.85 (ddd, 1H, J=7.8, 2.0, 1.3 Hz), 7.76 (m, 5H), 7.57 (t, 1H, J=8.0 Hz), 7.49 (dd, 2H, J=5.3, 2.8 Hz), 7.24 (d, 2H, J=8.8 Hz), 7.16 (m, 5H).

LCMS Rₜ 16.68 min.; purity 90.6%; MS m/z 451 [M]⁺.

1. A sulphonamide derivative of formula (I) or a physiologically acceptable salt thereof, 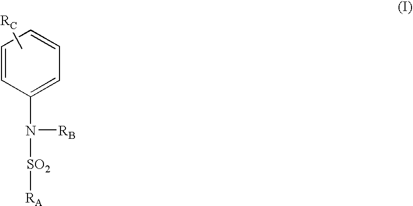
where
R_{C} is selected from a group consisting of dialkylamino, NO₂, CN, aminocarbonyl, monoalkylaminocarbonyl, dialkylaminocarbonyl, alkanoyl, oxazol-2-yl, oxazolylaminocarbonyl, aryl, aroyl, aryl-CH(OH)—, arylaminocarbonyl, furanyl, where the aryl, aroyl and furanyl moieties may be substituted, guanidinyl-(CH₂)_{z}—N(R′)—, Het-(CH₂)_{z}—N(R′)—, Het-CO—N(R′)—, Het-CH(OH)— and Het-CO—, where Het is an optionally substituted 46-membered heterocyclic ring containing one or more heteroatoms selected from N, S and O, R′ is hydrogen or alkyl, and z is an integer 1 to 5;
R_{A} is a group having the formula 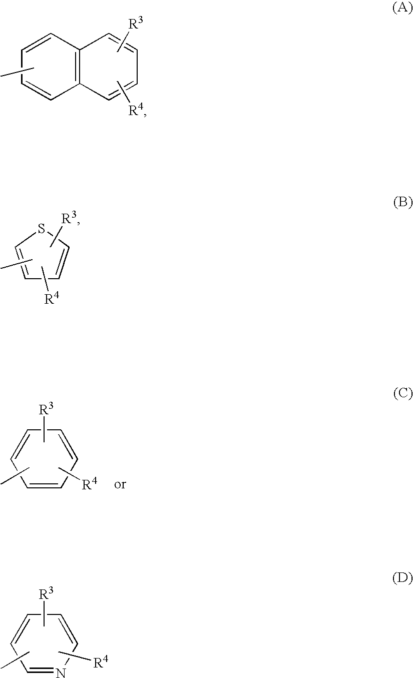
wherein
R³ and R⁴ represent each independently hydrogen, halogen, aryl, alkoxy, carboxy, hydroxy, alkoxyalkyl, alkoxycarbonyl, cyano, trifluoromethyl, alkanoyl, alkanoylamino, trifluoromethoxy, an optionally substituted aryl or heterocyclic group, and
R_{B} is hydrogen, alkyl, alkanoyl, hydroxyalkyl, alkoxyalkyl, alkoxycarbonyl, alkoxycarbonylalkyl, aminoalkyl, mono- or dialkylaminoalkyl or Het-alkyl, where Het is as defined above;
provided that
(i) when R_{C} is dialkylamino, then R_{B} is not hydrogen or alkyl;
(ii) when R_{A} is a group of formula (C), where R³ is hydrogen and R⁴ is methoxy, then R_{C} is not Het-CO—N(R′)—; and
(iii) when R_{A} is a group of formula (C), where R³ and R⁴ are hydrogen or halogen, then R_{C} is not nitro.

2. A derivative according to claim 1 where R_{C} is benzoyl.

3. A derivative according to claim 1 where R_{C} is α-hydroxybenzyl.

4. A derivative according to claim 1 where R_{C} is 2-oxo-imidazolidine-1-carbonyl-methyl-amino.

5. A derivative according to any of claims 1 to 4, where R_{A} is a group of formula (C), where R³ is chloro and R⁴ is chloro.

6. A derivative according to any of claims 1 to 4, where R_{A} is a group of formula (C), where R³ is hydrogen and R⁴ is 4-fluorophenyl.

7. A derivative according to any of claims 1 to 6, where R_{B} is alkyl.

8. A derivative according to claim 1, which is 4′-fluoro-biphenyl-3-sulfonic acid (4-benzoyl-phenyl)-amide.

9. A derivative according to claim 1, which is 4′-fluoro-biphenyl-3-sulfonic acid (3-benzoyl-phenyl)-amide.

10. A derivative according to claim 1, which is 4′-fluoro-biphenyl-3-sulfonic acid (α-hydroxybenzyl-phenyl)-amide.

11. A derivative according to claim 1, which is 2-oxo-imidazolidine-1-carboxylic acid {4-[(4′-fluoro-biphenyl-3-sulfonyl)-methyl-amino]-phenyl}-amide.

12. A derivative according to any of claims 1 to 11 for use as an inhibitor for collagen receptor integrins.

13. A derivative according to any of the claims 1 to 11 for use as an inhibitor for α2β1 integrin.

14. A derivative according to any of claims 1 to 11 for use as an α2β1 integrin I domain inhibitor.

15. A derivative according to any of claims 1 to 11 or a physiologically acceptable salt thereof for use as a medicament.

16. A derivative according to claim 15 for use as a medicament for treating thrombosis, inflammation, cancer and vascular diseases.

17. The use of a derivative according to any of claims 1 to 11 or a physiologically acceptable salt thereof for preparing a pharmaceutical composition for treating disorders relating to thrombosis, inflammation, cancer and vascular diseases.

18. A pharmaceutical composition comprising an effective amount of a derivative according to any of claims 1 to 11 or a physiologically acceptable salt thereof in admixture with a pharmaceutically acceptable carrier.

19. A process for preparing a sulphonamide according to claim 1, comprising reacting a compound of formula (II) 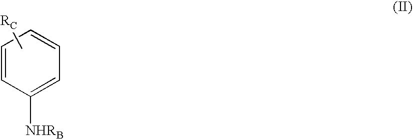
where R_{B} and R_{C} are as defined above, with a compound of formula (III)
R_{A}—SO₂hal (III)
where R_{A} is as defined above and hal is halogen.
